(19) United States
(12) Patent Application Publication
(10) Pub. No.: US 2011/0295139 A1
(43) Pub. Date: Dec. 1, 2011

(54) Method and system for reliable respiration parameter estimation from acoustic physiological signal

(76) Inventors: Te-Chung Isaac Yang, Aliso Viejo, CA (US); Yongji Fu, Vancouver, WA (US)

(21) Appl. No.: 12/802,044

(22) Filed: May 28, 2010

(51) Int. Cl. A61B 5/08

(52) U.S. Cl. 600/529

(57) ABSTRACT

A method and system that reliably estimates a respiration parameter from an acoustic physiological signal without introducing undue complexity or intense computation. A median filter is applied to an energy envelope of the signal to remove heart sound “sparks” from the envelope and better isolate lung sounds. The median filter is followed by a low-pass filter that removes abrupt changes in the envelope caused by the median filter's nonlinearity. Various peak cross-checks are performed on an autocorrelation result generated from the envelope to confirm the reliability of the signal before an estimate of a respiration parameter is generated from the autocorrelation result.

### BACKGROUND OF THE INVENTION

The present invention relates to respiration parameter estimation and, more particularly, to a method and system for reliably estimating respiration parameters from an acoustic physiological signal.

Real-time monitoring of the physiological state of people who suffer from chronic diseases is an important aspect of chronic disease management. Real-time physiological monitoring is in widespread use in managing cardiovascular, pulmonary and respiratory disease, and is also widely used in other contexts such as elder care. Many real-time physiological monitoring devices monitor the physiological state of human subjects by detecting and evaluating acoustic signals that contain body sounds.

One problem encountered in real-time acoustic physiological monitoring is parameter estimation error caused by noise and unwanted information that infects the acoustic physiological signal. Real-time acoustic physiological monitoring is often performed using a portable (e.g. wearable) device that continually analyzes an acoustic physiological signal captured by a sound transducer positioned on the body, such as the trachea or back, as a person goes about his or her daily life. The captured signal includes lung sound, heart sound and noise from body movement and the surrounding environment. Before the captured signal can be used to accurately estimate a physiological parameter, noise and unwanted information must be removed from the signal or reduced to a great extent. Otherwise, the result will be erroneous estimation of physiological parameters by the device and outputting of erroneous estimates. Reliance on erroneous estimates can have serious adverse consequences on the health of the person being monitored. For example, erroneous estimates can lead the person being monitored or his or her clinician to improperly interpret physiological state and cause the person to undergo treatment that is not medically indicated, or forego treatment that is medically indicated.

One physiological parameter commonly monitored in real-time acoustic physiological monitoring is respiration rate. Before an acoustic physiological signal can be used to accurately estimate respiration rate, heart sounds and background noise must be removed from the signal or greatly reduced in order to isolate lung sounds. Known techniques for removing or reducing heart sounds and background noise from an acoustic physiological signal have relied on various combinations of bandpass filters, adaptive filters and wavelet transform. These techniques have been complex, computationally intensive and/or failed to adequately isolate lung sounds.

### SUMMARY OF THE INVENTION

The present invention provides a method and system that reliably estimate a respiration parameter from an acoustic physiological signal without introducing undue complexity or intense computation. In one inventive feature, a median filter is applied to an energy envelope of the signal to remove heart sound “sparks” from the envelope and better isolate lung sounds. The median filter is followed by a low-pass filter that removes abrupt changes in the envelope caused by the median filter's nonlinearity. In another inventive feature, various peak cross-checks are performed on an autocorrelation result generated from the envelope to confirm the reliability of the signal before an estimate of a respiration parameter is generated from the autocorrelation result.

In one aspect of the invention, a respiratory monitoring system comprises a transducer; a processor communicatively coupled with the transducer; and an output interface, wherein under control of the processor the system extracts an energy envelope of an acoustic physiological signal captured by the transducer, applies a median filter and a low-pass filter in sequence to the energy envelope, and generates an estimate of a respiration parameter using the sequentially filtered energy envelope, and wherein the estimate is outputted on the output interface.

In some embodiments, the system applies a band-pass filter to the signal before extracting the energy envelope.

In some embodiments, the system extracts the energy envelope using a standard deviation method.

In some embodiments, the system determines an autocorrelation result using the sequentially filtered energy envelope and makes the estimate using the autocorrelation result.

In some embodiments, the system validates the autocorrelation result using peak cross-checks before generating the estimate.

In some embodiments, the peak cross-checks comprise a determination that a time delay of a highest peak of the autocorrelation result conforms with a time delay of a second highest peak of the autocorrelation result.

In some embodiments, the peak cross-checks comprise a determination that a time delay of a highest peak of the autocorrelation result conforms with a previous estimate of a respiration parameter.

In some embodiments, the peak cross-checks comprise a determination that a time delay of a second highest peak of the autocorrelation result conforms with a previous estimate of a respiration parameter.

In some embodiments, the peak cross-checks comprise a determination that a time delay of a peak of the autocorrelation result conforms with a time delay of a peak of a previous autocorrelation result.

In some embodiments, the respiration parameter is respiration rate.

In some embodiments, the output interface comprises a user interface and the estimate is displayed on the user interface.

In some embodiments, the system is portable.

In another aspect of the invention, a respiratory monitoring system comprises a transducer; a processor communicatively coupled with the transducer; and an output interface, wherein under control of the processor the system extracts an energy envelope of an acoustic physiological signal captured by the transducer, determines an autocorrelation result using the energy envelope, validates the autocorrelation result using peak cross-checks and generates an estimate of a respiration parameter using the validated autocorrelation result, and wherein the estimate is outputted on the output interface.

In yet another aspect of the invention, a respiratory monitoring method comprises the steps of capturing by a physiological monitoring system an acoustic physiological signal; extracting by the system an energy envelope of the signal; applying by the system a median filter and a low-pass filter in sequence to the energy envelope; determining by the system an autocorrelation result using the sequentially filtered energy envelope; validating by the system the autocorrelation result using peak cross-checks; generating by the system an estimate of a respiration parameter using the validated autocorrelation result; and outputting by the system the estimate.

These and other aspects of the invention will be better understood by reference to the following detailed description taken in conjunction with the drawings that are briefly described below. Of course, the invention is defined by the appended claims.

### BRIEF DESCRIPTION OF THE DRAWINGS

FIG. 1 shows a respiratory monitoring system in some embodiments of the invention.

FIG. 2 shows a respiratory monitoring method in some embodiments of the invention.

FIG. 3 shows energy envelopes extracted from an acoustic physiological signal before and after application of a median filter, in some embodiments of the invention.

FIG. 4 shows energy envelopes extracted from an acoustic physiological signal before application of a median filter, after application of a median filter, and after application of a low-pass filter, in some embodiments of the invention.

FIG. 5 shows a method for validating an autocorrelation result of an acoustic physiological signal using peak cross-checks in some embodiments of the invention.

FIG. 6 shows an exemplary cross-check between the two highest peaks of a current sampling window.

FIGS. 7 and 8 show an exemplary cross-check between a highest peak of a current sampling window and a previous respiratory rate estimate.

FIGS. 9 and 10 show an exemplary cross-check between a second highest peak of a current sampling window and previous respiratory rate estimate.

FIGS. 11 and 12 show an exemplary cross-check between a second highest peak of a current sampling window and a second highest peak of a previous sampling window.

### DETAILED DESCRIPTION OF A PREFERRED EMBODIMENT

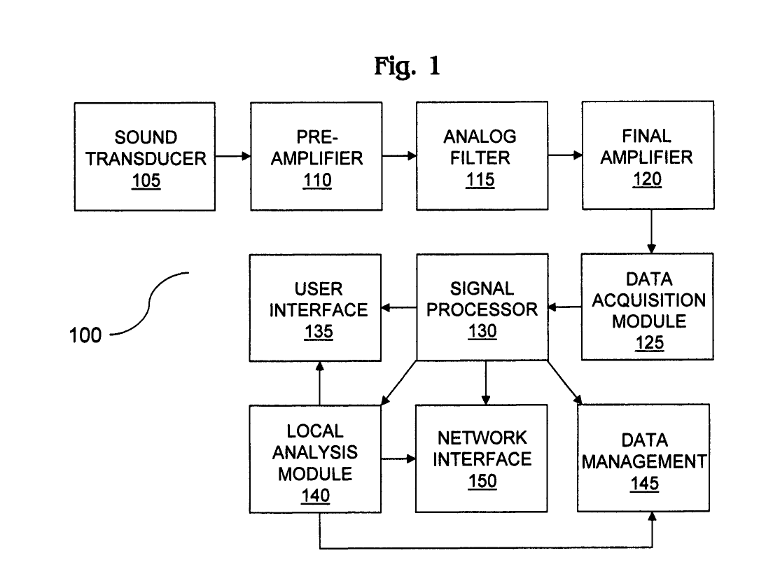

FIG. 1 shows a respiratory monitoring system 100 in some embodiments of the invention. System 100 includes a sound transducer 105 positioned on the body of a human subject being monitored. By way of example, transducer 105 may be positioned at a person's trachea, chest or back. Transducer 105 is communicatively coupled in series with a pre-amplifier 110, band-pass filter 115, final amplifier 120 and data acquisition module 125. Data acquisition module 125 transmits an acoustic physiological signal captured by transducer 105, as modified by amplifiers 110, 120 and filter 115, to a signal processor 130. Signal processor 130 processes the signal and outputs information generated from the signal to a user interface 135, a local analysis module 140, a data management element 145 and/or a network interface 150. The elements shown in FIG. 1, may be collocated or located remotely from one another. Adjacent elements shown in FIG. 1 may be communicatively coupled via wired or wireless links. In some embodiments, elements 105-150 are part of a wearable device that monitors a person's physiological state in real-time as the person performs daily activities.

Transducer 105 detects physiological sounds at a detection point, such as a person's trachea, chest or back. Transducer 105 in some embodiments is an omni-directional microphone housed in a chamber. Transducer 105 transmits captured sounds in the form of an electrical acoustic physiological signal to pre-amplifier 110 as analog voltages on the order of 10-200 mV.

Pre-amplifier 110 provides impendence match for the acoustic physiological signal received from transducer 105 and amplifies the signal to a level appropriate for the filter stage that follows.

Analog filter 115 is a coarse filter that applies a high-pass cutoff frequency at 80 Hz and a low-pass cutoff frequency at 2 KHz to the signal received from pre-amplifier 110 to reduce noise, such as muscle and contact noise. Final amplifier 120 amplifies the acoustic physiological signal received from filter 115 to the range of +/−1 V.

Data acquisition module 125 performs analog-digital (ND) conversion on the acoustic physiological signal received from amplifier 120 and transmits the signal to signal processor 130 for analysis. Data acquisition module 125 may also provide automatic gain control to adjust the amplitude of the signal provided to signal processor 130 without impacting on signal-to-noise ratio.

Signal processor 130 is a microprocessor having software executable thereon for performing signal processing on the acoustic physiological signal received from data acquisition module 125. Signal processing includes extracting an energy envelope from the signal, applying a median filter to the energy envelope to remove heart sound “sparks” from the envelope and better isolate lung sounds, applying a low-pass filter to the energy envelope to remove abrupt changes in the energy envelope caused by the median filter's nonlinearity, generating an autocorrelation result from the sequentially filtered energy envelope, performing various peak cross-checks on the autocorrelation result to confirm the reliability of the signal and generating an estimate of a respiration parameter, such as respiration rate, from the autocorrelation result.

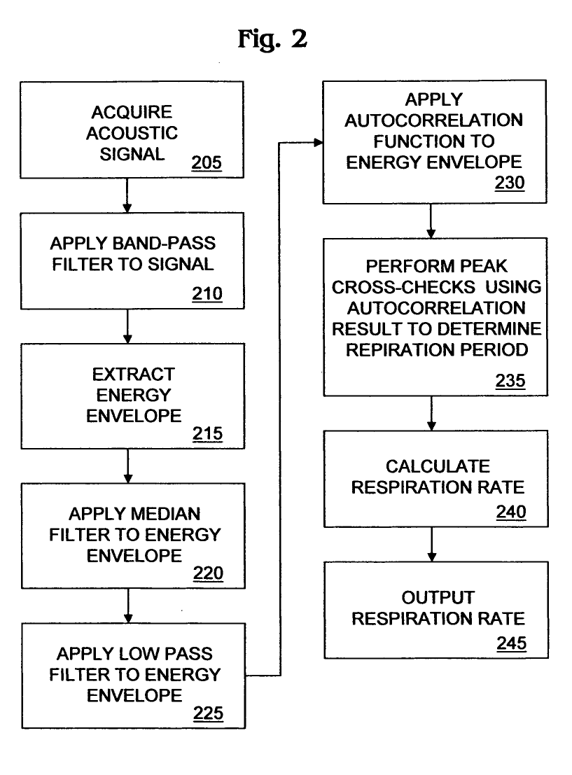

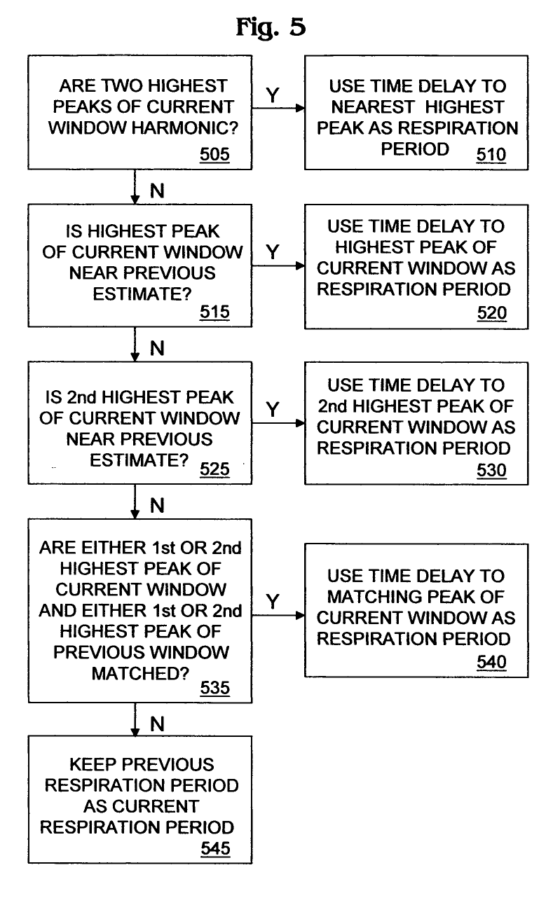

A respiratory monitoring method performed by system 100 under control of signal processor 130 will now be described by reference to the flow diagrams of FIGS. 2 and 5 taken in conjunction with the plots of FIGS. 3, 4 and 6-12. In the illustrated example, the goal of respiratory monitoring is to provide real-time estimates of respiration rate based on lung sounds detected at the trachea. However, it bears nothing that the method can be applied to achieve other respiratory monitoring goals, such as estimation of inspiration/expiration parameters, and can provide such estimates based on detection of body sounds elsewhere on the body, such as the chest or back.

In Step 205, an acoustic physiological signal is acquired by signal processor 130 from data acquisition module 125. The signal includes body sounds (heartbeat, breath sounds, etc.) intermingled with noise from different sources. Lung sounds are the body sounds of interest since the stated goal in the present example is to provide real-time estimates of respiratory rate. The signal is acquired in discrete segments of predetermined duration, such as 15 seconds. The discrete segments are sometimes called sampling windows herein.

In Step 210, a band-pass filter is applied to the acoustic physiological signal to isolate lung sounds by reducing heart sounds and further reducing noise. As lung sounds are typically found within the 300 Hz to 800 Hz frequency range, the band-pass filter may be a fifth order Butterworth filter having a high-pass cutoff frequency at 300 Hz and a low-pass cutoff frequency at 800 Hz.

In Step 215, an energy envelope is extracted from the acoustic physiological signal to further improve signal-to-noise ratio. In some embodiments, a standard deviation method is used to extract the energy envelope. In an exemplary standard deviation method, the standard deviation of every N consecutive samples, which is representative of the total energy of those N samples, is computed and used as envelope data. In FIG. 3, an energy envelope 310 extracted from an acoustic physiological signal using a standard deviation method is shown. Periodic lung sounds are clearly expressed in energy envelope 310. However, heart sounds persist in energy envelope 310 in the form of “sparks” on the lung sounds.

In Step 220 a median filter is applied to the acoustic physiological signal to better isolate lung sounds by removing the heart sound “sparks” from energy envelope 310. The median filter adopts the median of every N consecutive samples as the energy across the window of N samples, which has the effect of eliminating the heart sound “sparks.” In FIG. 3, an energy envelope 320 of the acoustic physiological signal after application of a median filter is shown. Heart sound “sparks” that were present in energy envelope 310 have been removed. However, the median filter, due to its nonlinear properties, introduces abrupt changes into energy envelope 320.

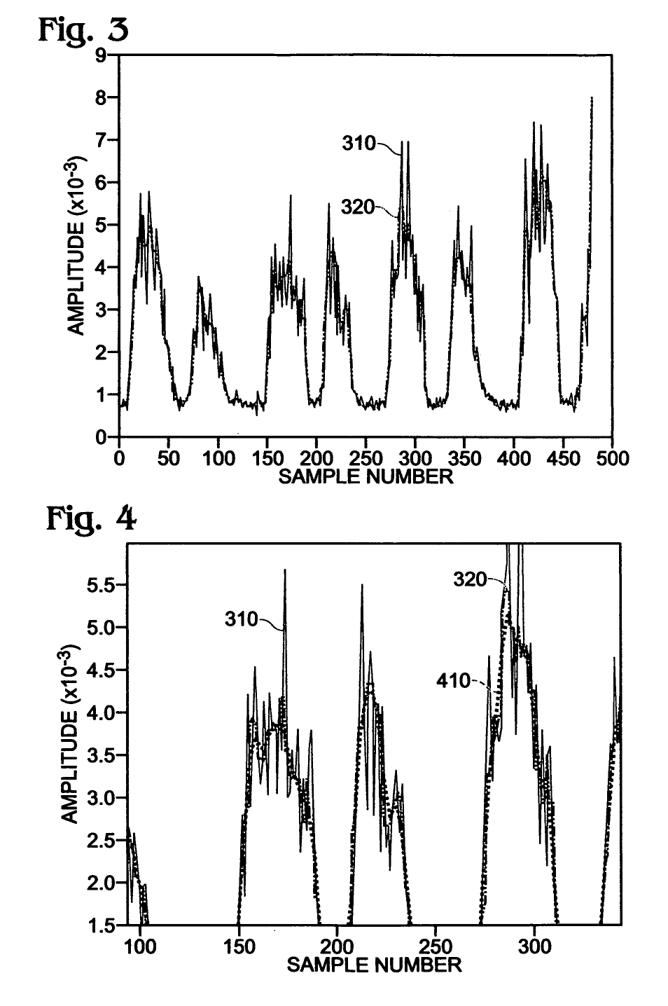

In Step 225, a low-pass filter is applied to the acoustic physiological signal to eliminate the abrupt changes introduced by the median filter. In FIG. 4, an energy envelope 410 of the acoustic physiological signal after application of the low-pass filter is shown. Abrupt changes present in energy envelope 320 have been removed.

In Step 230, an autocorrelation function is applied to energy envelope 330 to generate an autocorrelation result that identifies the fundamental periodicity in the acoustic physiological signal over the sampling window. The autocorrelation result is characterized by significant peaks including a central peak centered at zero time delay (t=0) and non-central peaks centered at positive time delays. Generally speaking, the time delay to a significant non-central peak is adopted as an estimate of respiration period from which signal processor 130 generates an estimate of respiration rate.

In Step 235, peak cross-checks are performed using the autocorrelation result generated in Step 230 to validate the result and ascertain a respiration period estimate. Turning to FIG. 5, a method for validating the current autocorrelation result using peak cross-checks in some embodiments of the invention is shown. In the discussion that follows, the highest peaks are chosen from among non-central peaks (i.e. peaks at positive time delays). The central peak is not selected.

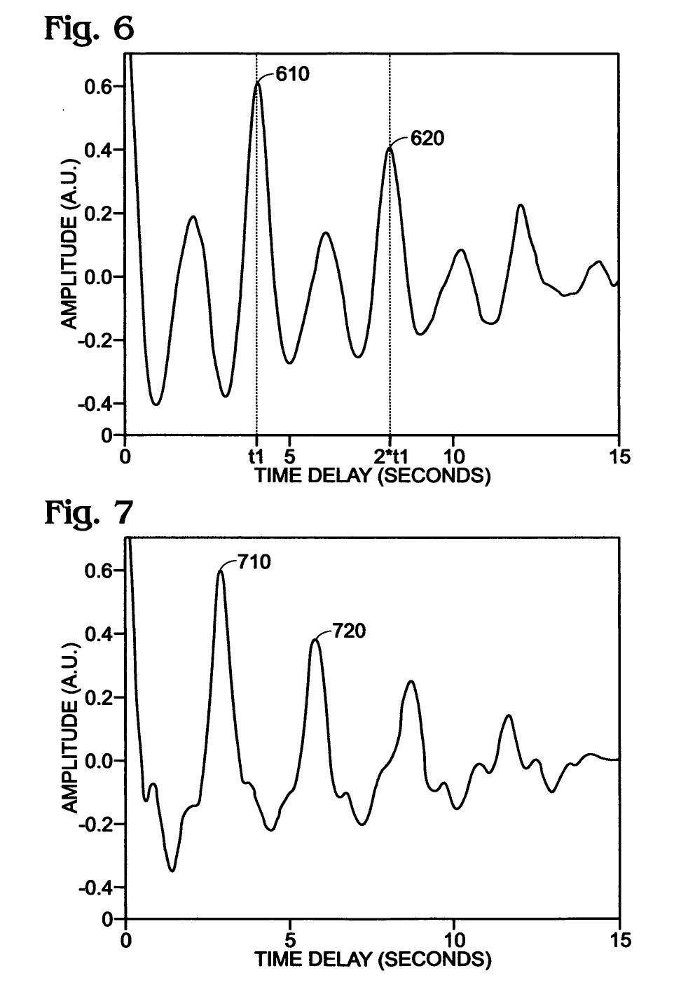

In Step 505, a cross-check is made whether the two highest peaks in the current sampling window (i.e. the current autocorrelation result) are harmonic. The two highest peaks are considered harmonic if the time delay between the central peak and the nearest one of the two highest peaks is approximately the same as the time delay between the nearest one of the two highest peaks and the other one of the two highest peaks. For example, in FIG. 6, a current sampling window is shown to have a highest peak 610 centered at a time delay t₁ and a second highest peak 620 centered at a time delay 2t₁. These peaks are thus considered harmonic. In some embodiments, the two highest peaks are considered harmonic if 1.9<t₂/t₁<2.1, where t₁ is the time delay from the central peak to the nearest one of the two highest peaks and t₂ is the time delay from the nearest one of the two highest peaks to the other one of the two highest peaks. If the two highest peaks of the current sampling window are harmonic, the flow proceeds to Step 510 and the time delay from the central peak to the nearest one of the two highest peaks (t₁) is adopted as the current respiration period estimate. If the two highest peaks of the current sampling window are not harmonic, further cross-checks are required to identify the current respiration period estimate and the flow proceeds to Step 515.

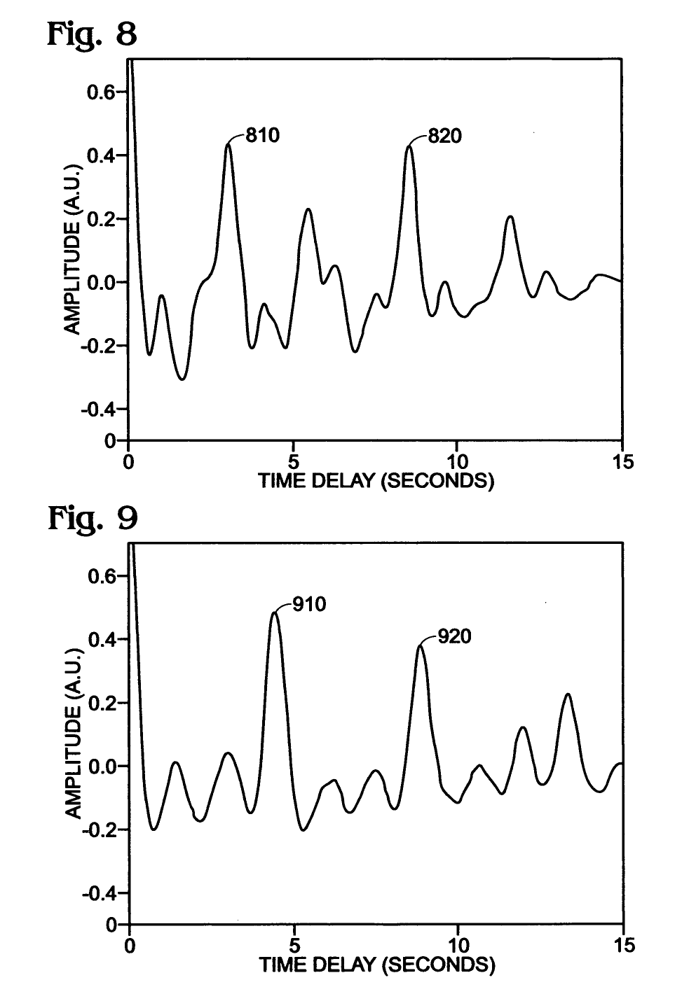

In Step 515, a cross-check is made whether the highest peak in the current sampling window is near the previous respiration period estimate. The highest peak is considered near the previous estimate if the time delay between the central peak and the highest peak in the current sampling window is approximately the same as the respiration period estimate acquired from the immediately preceding sampling window. For example, FIGS. 7 and 8 show contiguous sampling windows: a previous sampling window (FIG. 7) and a current sampling window (FIG. 8). In the previous sampling window, the two highest peaks 710, 720 are harmonic such that the previous respiration period estimate is taken as the time delay to the highest peak 710. In the current sampling window, however, the two highest peaks 810, 820 are not harmonic. However, the time delay to the highest peak 810 in the current sampling window is near the previous respiration period estimate, such that the cross-check of Step 515 is satisfied. In some embodiments, the time delay to the highest peak in the current sampling window is considered near the previous respiration period estimate if (t₂−t₁)/t₁<0.4, where t₂ is the time delay from the central peak to the highest peak in the current sampling window and t₁ is the previous respiration period estimate. If the time delay to the highest peak in the current sampling window is near the previous respiration period estimate, the flow proceeds to Step 520 and the time delay from the central peak to the highest peak in the current sampling window is adopted as the current respiration period estimate. If the time delay to the highest peak in the current sampling window is not near the previous respiration period estimate, further cross-checks are required to identify the current respiration period estimate and the flow proceeds to Step 525.

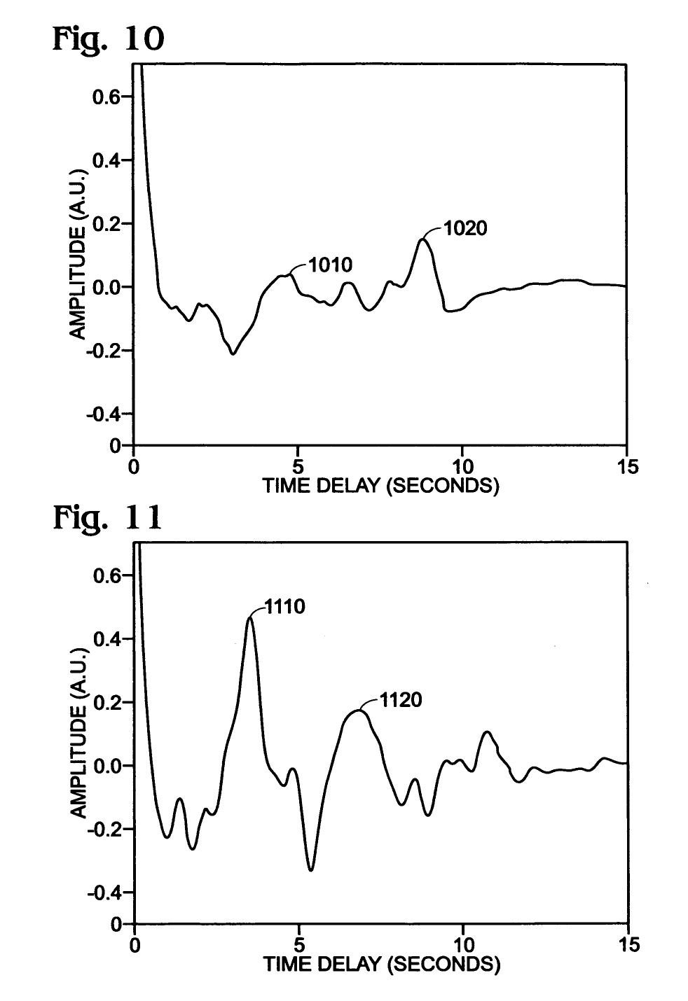

In Step 525, a cross-check is made whether the second highest peak in the current sampling window is near the previous respiration period estimate. The second highest peak is considered near the previous estimate if the time delay between the central peak and the second highest peak in the current sampling window is approximately the same as the respiration period estimate taken from the immediately preceding sampling window. For example, FIGS. 9 and 10 show contiguous sampling windows: a previous sampling window (FIG. 9) and a current sampling window (FIG. 10). In the previous sampling window, the two highest peaks 910, 920 are harmonic such that the previous respiration period estimate is taken as the time delay to the highest peak 910. In the current sampling window, however, the two highest peaks 1010, 1020 are not harmonic. Moreover, the time delay to the highest peak 1020 in the current sampling window is not near the previous respiration period estimate. However, the time delay to the second highest peak 1010 in the current sampling window is near the previous respiration period estimate, such that the cross-check of Step 525 is satisfied. In some embodiments, the time delay to the second highest peak in the current sampling window is considered near the previous respiration period estimate if (t₂−t₁)/t₁<0.4, where t₂ is the time delay from the central peak to the second highest peak in the current sampling window and t₁ is the previous respiration period estimate. If the time delay to the second highest peak in the current sampling window is near the previous respiration period estimate, the flow proceeds to Step 530 and the time delay from the central peak to the second highest peak in the current sampling window is adopted as the current respiration period estimate. If the time delay to the second highest peak in the current sampling window is not near the previous respiration period estimate, further cross-checks are required to identify the current respiration period estimate and the flow proceeds to Step 535.

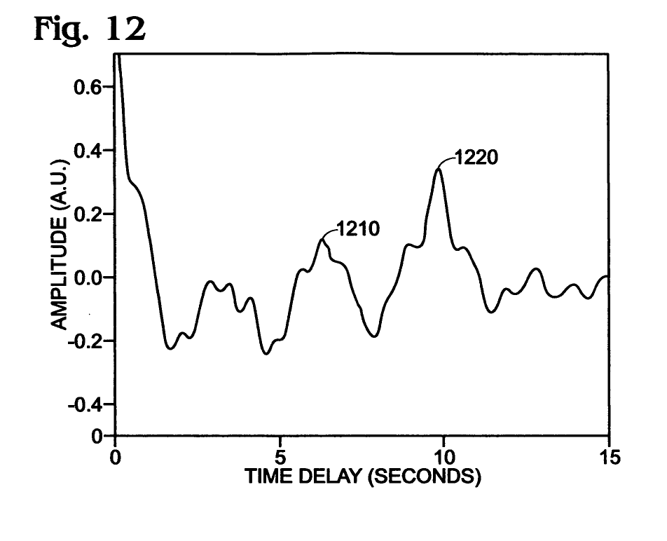

In Step 535, a cross-check is made whether either the first or second highest peak in the current sampling window matches either the first or second highest peak in the previous sampling window. For purposes of this cross-check, peaks are considered matching if the time delay between the central peak and the peak of interest in the current sampling window is approximately the same as the time delay between the central peak and the peak of interest in the immediately preceding sampling window. For example, FIGS. 11 and 12 show contiguous sampling windows: a previous sampling window (FIG. 11) and a current sampling window (FIG. 12). In the previous sampling window, the two highest peaks 1110, 1120 are harmonic such that the previous respiration period estimate is taken as the time delay to the highest peak 1110. In the current sampling window, however, the two highest peaks 1210, 1220 are not harmonic. Moreover, the time delay to the highest peak 1220 in the current sampling window is not near the previous respiration period estimate. Furthermore, the time delay to the second highest peak 1210 in the current sampling window is not near the previous respiration period estimate. However, the time delay between the central peak and the second highest peak 1210 in the current sampling window is approximately the same as the time delay between the central peak and the second highest peak 1120 in the immediately preceding sampling window, such that the cross-check of Step 535 is satisfied. In some embodiments, the peaks of interest are deemed to match if (t₂−t₁)/t₁<0.4, where t₂ is the time delay from the central peak to the peak of interest in the current sampling window and t₁ is the time delay from the central peak to the peak of interest in the previous sampling window. If either the first or second highest peak in the current sampling window matches either the first or second highest peak in the previous sampling window, the flow proceeds to Step 540 and the time delay from the central peak to the second highest peak in the current sampling window is adopted as the current respiration period estimate. If there is no match, the current autocorrelation result is not considered sufficiently reliable to form the basis of a respiration rate estimate and the flow proceeds to Step 545 where the previous respiration period estimate is maintained as the current respiration period estimate.

A record of the first and second highest peaks in each sampling window is stored under control of signal processor 130 in a peak data log for application, as needed, in cross-checks to determine respiration period estimates. Returning to FIG. 2, in Step 240, the current respiration period estimate is applied to generate a current respiration rate estimate according to the formula RR=60/RP, where RR is the respiration rate in breaths per minute and RP is the respiration period. Then, in Step 245, the respiration rate is outputted on one or more output interfaces. For example, the respiration rate estimate may be transmitted to user interface 135 whereon the respiration rate estimate is displayed to the person being monitored, transmitted to local analysis module 140 whereon the estimate is subjected to higher level clinical processing, transmitted to data management element 145 whereon the estimate is logged, and/or transmitted to network interface 150 for further transmission to a remote analysis module or remote clinician display. Moreover, if the current autocorrelation result was deemed insufficiently reliable to form the basis for a respiration rate estimate in Step 545, an alert may be outputted to/on one or more of elements 135, 140, 145, 150 to provide notice of the reliability problem.

It will be appreciated by those of ordinary skill in the art that the invention can be embodied in other specific forms without departing from the spirit or essential character hereof. The present description is therefore considered in all respects to be illustrative and not restrictive. The scope of the invention is indicated by the appended claims, and all changes that come with in the meaning and range of equivalents thereof are intended to be embraced therein.

What is claimed is:

1. A respiratory monitoring system, comprising:
a transducer;
a processor communicatively coupled with the transducer; and
an output interface, wherein under control of the processor the system extracts an energy envelope of an acoustic physiological signal captured by the transducer, applies a median filter and a low-pass filter in sequence to the energy envelope, and generates an estimate of a respiration parameter using the sequentially filtered energy envelope, and wherein the estimate is outputted on the output interface.

2. The system of claim 1, wherein under control of the processor the energy envelope is extracted using a standard deviation method.

3. The system of claim 1, wherein under control of the processor an autocorrelation result is determined using the sequentially filtered energy envelope and the estimate is made using the autocorrelation result.

4. The system of claim 3, wherein the autocorrelation result is validated using peak cross-checks before the estimate is generated.

5. The system of claim 4, wherein the peak cross-checks comprise a determination that a time delay of a highest peak of the autocorrelation result conforms with a time delay of a second highest peak of the autocorrelation result.

6. The system of claim 4, wherein the peak cross-checks comprise a determination that a time delay of a highest peak of the autocorrelation result conforms with a previous estimate of a respiration parameter.

7. The system of claim 4, wherein the peak cross-checks comprise a determination that a time delay of a second highest peak of the autocorrelation result conforms with a previous estimate of a respiration parameter.

8. The system of claim 4, wherein the peak cross-checks comprise a determination that a time delay of a peak of the autocorrelation result conforms with a time delay of a peak of a previous autocorrelation result.

9. The system of claim 1, wherein the respiration parameter is respiration rate.

10. The system of claim 1, wherein the output interface comprises a user interface and the estimate is displayed on the user interface.

11. The system of claim 1, wherein the system is portable.

12. A respiratory monitoring system, comprising:
a transducer;
a processor communicatively coupled with the transducer; and
an output interface, wherein under control of the processor the system extracts an energy envelope of an acoustic physiological signal captured by the transducer, determines a autocorrelation result using the energy envelope, validates the autocorrelation result using peak cross-checks and generates an estimate of a respiration parameter using the validated autocorrelation result, and wherein the estimate is outputted on the output interface.

13. The system of claim 12, wherein the peak cross-checks comprise a determination that a time delay of a highest peak of the autocorrelation result conforms with a time delay of a second highest peak of the autocorrelation result.

14. The system of claim 12, wherein the peak cross-checks comprise a determination that a time delay of a highest peak of the autocorrelation result conforms with a previous estimate of a respiration parameter.

15. The system of claim 12, wherein the peak cross-checks comprise a determination that a time delay of a second highest peak of the autocorrelation result conforms with a previous estimate of a respiration parameter.

16. The system of claim 12, wherein the peak cross-checks comprise a determination that a time delay of a peak of the autocorrelation result conforms with a time delay of a peak of a previous autocorrelation result.

17. The system of claim 12, wherein a median filter and a low-pass filter are applied in sequence to the energy envelope before the autocorrelation result is determined.

18. The system of claim 12, wherein the output interface comprises a user interface and the estimate is displayed on the user interface.

19. A respiratory monitoring method, comprising the steps of:
capturing by a physiological monitoring system an acoustic physiological signal;
extracting by the system an energy envelope of the signal;
applying by the system a median filter and a low-pass filter in sequence to the energy envelope;
determining by the system a autocorrelation result using the sequentially filtered energy envelope;
validating by the system the autocorrelation result using peak cross-checks;
generating by the system an estimate of a respiration parameter using the validated autocorrelation result; and
outputting by the system the estimate.

20. The method of claim 19, wherein the estimate is displayed on a user interface.
